(19) United States
(12) Patent Application Publication
(10) Pub. No.: US 2026/0148838 A1
(43) Pub. Date: May 28, 2026

(54) MEDICAL FOUNDATION MODEL FOR VARIABLE NUMBER OF 3D ANISOTROPIC MEDICAL IMAGES

(71) Applicant: Siemens Healthineers AG, Forchheim (DE)

(72) Inventors: Gengyan Zhao, Robbinsville, NJ (US); Badhan Kumar Das, Erlangen (DE); Boris Mailhe, Plainsboro, NJ (US); Bogdan Georgescu, Princeton, NJ (US); Yue Zhang, Short Hills, NJ (US); Long Xie, Chesterbrook, PA (US); Eli Gibson, Lawrenceville, NJ (US); Dorin Comaniciu, Princeton, NJ (US)

(21) Appl. No.: 18/961,736

(22) Filed: Nov. 27, 2024

(51) Int. Cl. G16H 30/40; G06T 7/00; G06V 10/75; G06V 10/82; G16H 50/20

(52) U.S. Cl. CPC G16H 30/40; G06T 7/0012; G06V 10/751; G06V 10/82; G16H 50/20; G06T 2200/04; G06T 2207/20084

(57) ABSTRACT

Systems and methods for performing a medical imaging analysis task using a foundation model are provided. One or more 3D (three-dimensional) medical images each comprising a plurality of 2D (two-dimensional) slices are received. A first set of features is extracted from the plurality of 2D slices of the one or more 3D medical images using a machine learning based encoding network. Each respective feature of the first set of features is resampled based on a spatial location of one or more pixels of the 2D slices from which the respective feature was extracted. The resampled first set of features is encoded into a second set of features. A medical imaging analysis task is performed based on the second set of features. Results of the medical imaging analysis task are output.

### TECHNICAL FIELD

The present invention relates generally to AI/ML (artificial intelligence/machine learning) based medical imaging analysis, and in particular to a medical foundation model for a variable number of 3D anisotropic medical images.

### BACKGROUND

Recently, the advancement of foundation models has garnered significant attention, with notable progress observed in various applications. For instance, LLM (large language model) advancement has significantly improved question answering tasks, while diffusion models have excelled in image synthesis. However, the inherent complexity and heterogeneity of medical imaging data present significant challenges in the creation of a foundation model.

Conventional foundation models in the medical imaging field typically accept a single image with fixed dimensions. However, medical diagnoses often necessitate the utilization of multiple input medical images, such as multiple MR (magnetic resonance) images with different contrasts with varying anisotropic resolutions. Moreover, the number of input images and the corresponding contrasts acquired may vary across different diagnostic tasks and clinical sites.

### BRIEF SUMMARY OF THE INVENTION

In accordance with one or more embodiments, systems and methods for performing a medical imaging analysis task using a foundation model are provided. One or more 3D (three-dimensional) medical images each comprising a plurality of 2D (two-dimensional) slices are received. A first set of features is extracted from the plurality of 2D slices of the one or more 3D medical images using a machine learning based encoding network. Each respective feature of the first set of features is resampled based on a spatial location of one or more pixels of the 2D slices from which the respective feature was extracted. The resampled first set of features is encoded into a second set of features. A medical imaging analysis task is performed based on the second set of features. Results of the medical imaging analysis task are output.

In one embodiment, the one or more 3D input medical images are of at least one of different resolutions, different acquisition orientations, or different domains.

In one embodiment, each respective feature of the first set of features is resampled onto a 2D uniform pixel grid to form 2D images in a feature space. The 2D images in the feature space are stacked to form one or more 3D isotropic images in the feature space.

In one embodiment, the spatial location of the one or more pixels of the 2D slices comprises coordinates of the one or more pixels.

In one embodiment, the first set of features are encoded with positional embeddings and/or modality embeddings.

In one embodiment, the resampled first set of features is encoded into the second set of features using at least one of a transformer encoder or a state space model.

In one embodiment, the medical imaging analysis task is performed using at least one of a transformer encoder or a state space model.

These and other advantages of the invention will be apparent to those of ordinary skill in the art by reference to the following detailed description and the accompanying drawings.

### BRIEF DESCRIPTION OF THE DRAWINGS

FIG. 1 shows a method for performing a medical imaging analysis task using a medical imaging foundation model, in accordance with one or more embodiments;

FIG. 2 shows a workflow for performing a medical imaging analysis task using a medical imaging foundation model, in accordance with one or more embodiments;

FIG. 3 shows an exemplary artificial neural network that may be used to implement one or more embodiments;

FIG. 4 shows a convolutional neural network that may be used to implement one or more embodiments;

FIG. 5 shows a data flow diagram of a generative adversarial network that may be used to implement one or more embodiments;

FIG. 6 shows a schematic structure of a recurrent machine learning model that may be used to implement one or more embodiments; and

FIG. 7 shows a high-level block diagram of a computer that may be used to implement one or more embodiments.

### DETAILED DESCRIPTION

The present invention generally relates to methods and systems for performing a medical imaging analysis task using a medical foundation model for variable number of 3D anisotropic medical images. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a novel foundation model framework, which can efficiently handle a varying number of heterogeneous input medical images with different anisotropic resolutions, different acquisition orientations, and/or different domains. The framework eliminates the need for training multiple models or encoders to accommodate different numbers of input medical images and obviates the requirement for extensive up/down sampling of the input medical images with distinct anisotropic resolutions. The foundation model in accordance with embodiments described herein can take advantage of all available input data with minimal preprocessing, and the pretrained foundation model can be leveraged to address various downstream tasks with disparate requirements and varying numbers of input medial images.

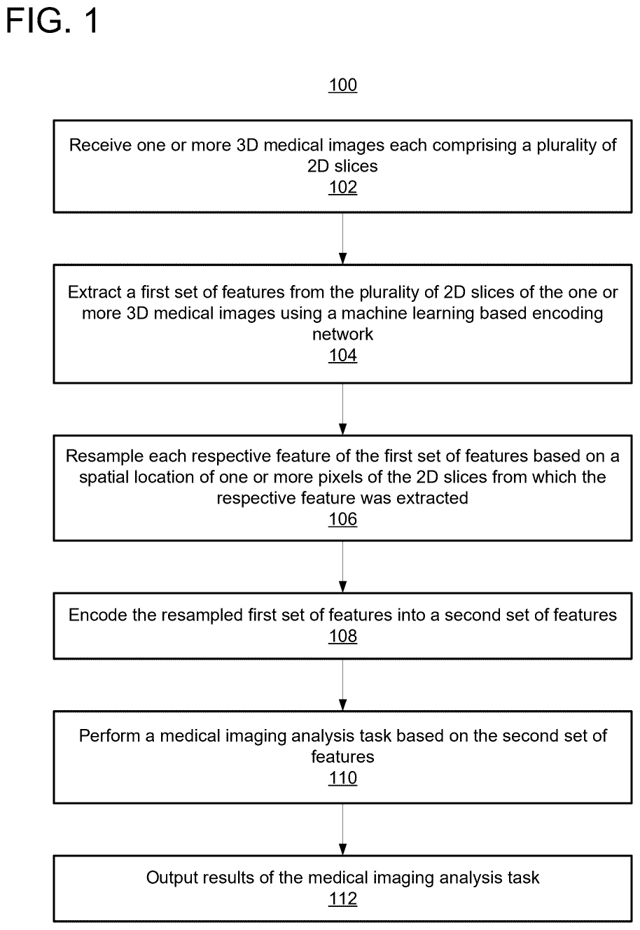

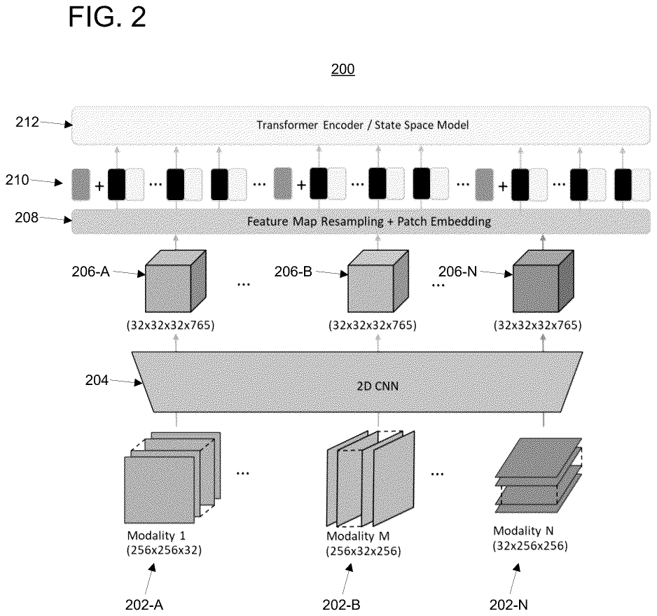

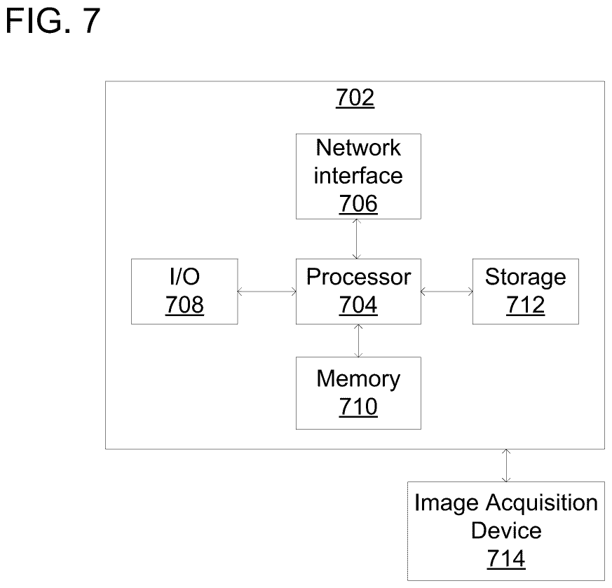

FIG. 1 shows a method 100 for performing a medical imaging analysis task using a medical imaging foundation model, in accordance with one or more embodiments. The steps and sub-steps of method 100 may be performed by one or more suitable computing devices, such as, e.g., computer 702 of FIG. 7. FIG. 2 shows a workflow 200 for performing a medical imaging analysis task using a medical imaging foundation model, in accordance with one or more embodiments. FIG. 1 and FIG. 2 will be described together.

At step 102 of FIG. 1, one or more 3D (three-dimensional) medical images each comprising a plurality of 2D (two-dimensional) slices are received. In one example, as shown in workflow 200 of FIG. 2, the one or more 3D medical images is 3D medical images 202-A, 202-B, . . . , 202-N (collectively referred to as 3D medical images 202). 3D medical images 202 comprise 32 2D slices, each with a resolution of 256×256 pixels. The one or more 3D medical images may be anisotropic medical images depicting any anatomical object of interest of a patient.

In one embodiment, the one or more 3D medical images are of different resolutions, acquisition orientations, and/or domains. Acquisition orientations refer to different planes or angles from which the one or more 3D medical images are acquired to view various cross-sections of the body of the patient. Examples of acquisition orientations include axial, coronal, and sagittal orientations. As used herein, a domain of a medical image refers to the modality of the medical image as well as the protocol used for obtaining the medical image in that modality. The modality of the one or more input medical images may include, for example, MRI (magnetic resonance imaging), CT (computed tomography), US (ultrasound), x-ray, SPECT (single-photon emission computed tomography), PET (positron emission tomography), or any other medical imaging modality or combinations of medical imaging modalities. The protocol used for obtaining the medical image may include, for example, acquisition sequences or techniques for acquiring a medical image, such as, e.g., T1-weighted, T2-weighted, proton density-weighted MRI images, contrast and non-contrast images, CT images captured with low kV (kilovoltage) and high kV, or low and high resolution medical images. Accordingly, the domains may be completely different medical imaging modalities or different image protocols within the same overall imaging modality. The one or more input medical images may be represented in the image space (e.g., as pixel or voxel values in spatial coordinates) or the latent space (e.g., as a lower-dimensional, compressed representation of the one or more medical images represented as a feature vector).

The one or more 3D medical images may be received, for example, by directly receiving the one or more 3D medical images from an image acquisition device (e.g., image acquisition device 714 of FIG. 7) as the one or more 3D medical images are acquired, by loading the one or more 3D medical images from a storage or memory of a computer system (e.g., storage 712 or memory 710 of computer 702 of FIG. 7), or by receiving the one or more 3D medical images from a remote computer system (e.g., computer 702 of FIG. 7). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, a first set of features is extracted from the plurality of 2D slices of the one or more 3D medical images using a machine learning based encoding network.

In one embodiment, the machine learning based encoding network is a 2D CNN (convolutional neural network), such as, e.g., a 2D UNet. For example, as shown in workflow 200 of FIG. 2, features 206-A, 206-B, . . . , 206-N (collectively referred to as features 206) are respectively extracted from 3D medical images 202-A, 202-B, . . . , 202-N by 2D CNN 204. However, the machine learning based encoding network may be implemented according to any other suitable machine learning based architecture. The machine learning based encoding network receives as input each of the 2D slices of the one or more 3D medical images and generates as output the first set of features. The features of the first set of features are lower-dimensional, compressed representations of the 2D slices represented as feature vectors.

The machine learning based encoding network down-samples the 2D slices by progressively reducing the spatial resolution while capturing and encoding the most important features in a compressed, lower-dimensional representation. For example, as shown in workflow 200 of FIG. 2, 2D CNN 204 down-samples 2D slices of 3D medical images 202 to a resolution of 32×32×32 in the feature space with a total of 765 features.

At step 106 of FIG. 1, each respective feature of the first set of features is resampled based on a spatial location of one or more pixels of the 2D slices from which the respective feature was extracted. In one example, as shown in workflow 200 of FIG. 2, features 206 are resampled at feature map resampling block 208 to generate resampled features 210.

The features are resampled to align to the spatial location of one or more pixels of the 2D slices from which the features were extracted. The features are resampled onto a 2D uniform pixel grid to form 2D slices in the feature space of the one or more 3D medical images. The 2D slices/images in the feature space are stacked to form an isotropic 3D image in the feature space. For example, in workflow 200 of FIG. 2, features 206 are resampled on a 2D uniform pixel grid to form 32 2D slices in the image space and the 2D slices are stacked to form 3D isotropic images in the feature space. The resampling thus provides for isotropic 3D images in the feature space of the anisotropic one or more 3D medical images in the image space. The resampled first set of features generated by the machine learning based encoding network from different anisotropic images result in more similar and more isotropic resolutions, since the high-resolution dimensions are extracted to high-level features with lower resolutions. The resampling does not incur significant information loss since the anisotropic level is reduced when the 2D slices are down-sampled.

In one embodiment, the spatial location comprises coordinates (e.g., in the coordinate system of the image acquisition device) of each pixel of the 2D slices of the one or more 3D medical images. The coordinates may be generated at acquisition by the image acquisition device and stored in the header of the one or more 3D medical images.

In one embodiment, the resampled features are encoded with positional embeddings (e.g., sinusoidal or learnable) based on the spatial location of one or more pixels of the 2D slices from which the features were extracted. Thus, features of different 3D medical images but located at the same relative location within a 3D medical image will have the same positional embedding. The position embeddings help preserve spatial relationships of the features. In one embodiment, the resampled features are additionally or alternatively encoded with modality embeddings. The position embeddings and/or modality embeddings may be fixed or trainable. The modality embeddings may also be derived from the domain (e.g., the modality or contrast name) using a language encoder.

At step 108 of FIG. 1, the resampled first set of features is encoded into a second set of features. In one embodiment, the resampled first set of features is encoded using a machine learning based encoder network, such as, e.g., a transformer encoder. In another embodiment, the resampled first set of features is encoded using a state space model. For example, as shown in workflow 200 of FIG. 2, transformer encoder/state space model 212 encodes features 210 to generate additional features (not shown in FIG. 2). However, the resampled first set of features may be encoded into the second set of features using any other suitable approach. The machine learning based encoder network/state space model receives as input the resampled first set of features and generates as output the second set of features.

At step 110 of FIG. 1, a medical imaging analysis task is performed based on the second set of features. In one embodiment, the medical imaging analysis task may be performed using one or more machine learning based task networks, such as, e.g., a task-specific decoder head (e.g., a transformer decoder). The machine learning based task network receives as input the second set of features and generates as output results of the medical imaging analysis task. In another embodiment, the medical imaging analysis task may be performed using a state space model.

In one embodiment, the medical imaging analysis task is image synthesis for generating a synthetic image from the one or more medical images. The synthetic image may be in a domain, acquisition orientation, or resolution that is absent from the one or more 3D medical images. The medical imaging analysis task may additionally or alternatively comprise any other suitable task, such as, e.g., detection, segmentation, classification, quantification, etc.

At step 114 of FIG. 1, results of the medical imaging analysis task are output. For example, the results of the medical imaging analysis task can be output by displaying the results on a display device of a computer system (e.g., 1/O 708 of computer 702 of FIG. 7), storing the results on a memory or storage of a computer system (e.g., memory 710 or storage 712 of computer 702 of FIG. 7), or by transmitting the results to a remote computer system (e.g., computer 702 of FIG. 7).

Advantageously, embodiments described herein address the challenge of computational cost by implementing self-attention masks for transformers or by employing state space models such as, e.g., Mamba. Given that the foundation model in accordance with embodiments described herein accommodates multiple input medical images, the token length can be substantial. Utilizing a state space model may provide advantageous in significantly mitigating computational costs. Alternatively, where a transformer architecture is adopted, masked self-attention can be applied. Specifically, self-attention computation occurs among all tokens within two specific dimensions of each input medical image and among all tokens in the same single dimension across all images. This approach effectively optimizes computational efficiency while maintaining the model's ability to capture relevant interactions among tokens.

In the pretraining phase, self-supervised learning employing the masked auto-encoder strategy may be employed. Subsequently, for downstream tasks (i.e., for performing the medical imaging analysis task), the transformer or state space model is connected to specific decoders tailored to each task type, e.g. a classification head or a segmentation head. To enhance training robustness, conventional augmentation techniques such as random dropping input modalities may be implemented. Additionally, random sampling of a subset of input medical images or total tokens per case/subject is utilized to alleviate GPU memory constraints, thereby optimizing computational resources.

In one example, the one or more machine learning based task networks comprise a linear decoder. For instance, CMR (cardiac MR) is the gold standard for estimating ejection fraction, while cardiac ultrasound (or echocardiography, Echo) offers real-time imaging with live quantification and analysis. The two modalities are often acquired in different orientations. By combining these two modalities, diagnostic accuracy can be improved, particularly in detecting heart anomalies. In this scenario, input 3D medical images modalities such as, e.g., CMR and Echo are received at step 102 and output through a linear decoder at step 110, producing a score that indicates whether heart activity is normal or abnormal.

In addition to its diagnostic applications, embodiments described herein can also be employed during interventions. In one example, the one or more machine learning based task network comprise a multitask decoder. For example, during an LAA (left atrial appendage) closure procedure, both cardiac CT and live intracardiac echocardiography are often available to estimate the LAA ostium size, which determines the appropriate implant device. In this case, two decoders may be utilized, each translating the feature map into a segmentation map for the LAA ostium in its respective modality. The system functions as a registration engine, ensuring consistent anatomical segmentation across different modalities. This enables the physician to accurately assess anatomical changes by comparing pre-operative CT images with live echocardiography images.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the machine learning based encoding network utilized at steps 104 or 108 or the machine learning based decoding network utilized at step 110 of FIG. 1 or 2D CNN 204 or transformer encoder/state space model 212 of FIG. 2, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

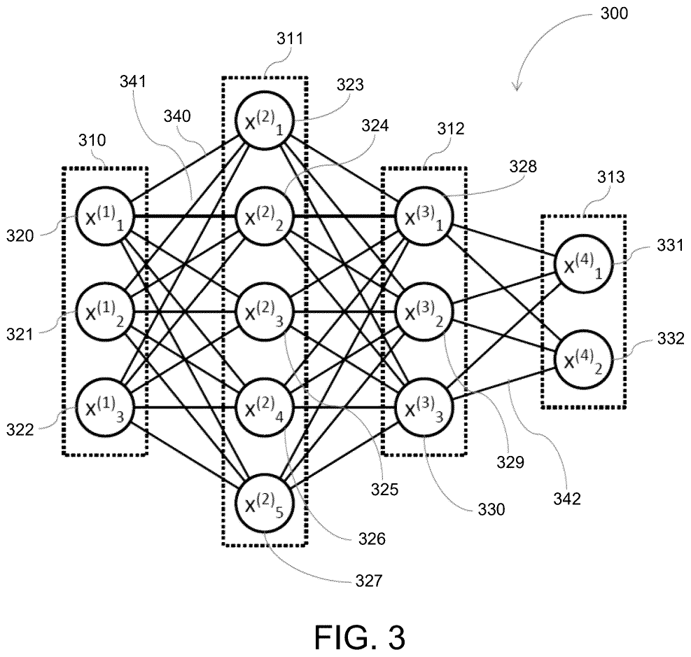

FIG. 3 shows an embodiment of an artificial neural network 300 that may be used to implement one or more machine learning models described herein. Alternative terms for “artificial neural network” are “neural network”, “artificial neural net” or “neural net”.

The artificial neural network 300 comprises nodes 320, . . . , 332 and edges 340, 342, wherein each edge 340, . . . , 342 is a directed connection from a first node 320, 332 to a second node 320, . . . , 332. In general, the first node 320, . . . , 332 and the second node 320, . . . , 332 are different nodes 320, . . . , 332, it is also possible that the first node 320, . . . , 332 and the second node 320, . . . , 332 are identical. For example, in FIG. 3 the edge 340 is a directed connection from the node 320 to the node 323, and the edge 342 is a directed connection from the node 330 to the node 332. An edge 340, . . . , 342 from a first node 320, . . . , 332 to a second node 320, . . . , 332 is also denoted as “ingoing edge” for the second node 320, . . . , 332 and as “outgoing edge” for the first node 320, . . . 332.

In this embodiment, the nodes 320, . . . , 332 of the artificial neural network 300 can be arranged in layers 310, . . . , 313, wherein the layers can comprise an intrinsic order introduced by the edges 340, . . . , 342 between the nodes 320, . . . , 332. In particular, edges 340, . . . , 342 can exist only between neighboring layers of nodes. In the displayed embodiment, there is an input layer 310 comprising only nodes 320, . . . , 322 without an incoming edge, an output layer 313 comprising only nodes 331, 332 without outgoing edges, and hidden layers 311, 312 in-between the input layer 310 and the output layer 313. In general, the number of hidden layers 311, 312 can be chosen arbitrarily. The number of nodes 320, . . . , 322 within the input layer 310 usually relates to the number of input values of the neural network, and the number of nodes 331, 332 within the output layer 313 usually relates to the number of output values of the neural network.

In particular, a (real) number can be assigned as a value to every node 320, . . . , 332 of the neural network 300. Here, x⁽ⁿ⁾i denotes the value of the i-th node 320, . . . , 332 of the n-th layer 310, . . . , 313. The values of the nodes 320, . . . , 322 of the input layer 310 are equivalent to the input values of the neural network 300, the values of the nodes 331, 332 of the output layer 313 are equivalent to the output value of the neural network 300. Furthermore, each edge 340, . . . , 342 can comprise a weight being a real number, in particular, the weight is a real number within the interval [−1, 1] or within the interval [0, 1]. Here, w^{(m,n)}i,j denotes the weight of the edge between the i-th node 320, . . . , 332 of the m-th layer 310, . . . , 313 and the j-th node 320, . . . , 332 of the n-th layer 310, . . . , 313. Furthermore, the abbreviation w⁽ⁿ⁾i,j is defined for the weight w^{(n,n+1)}i,j.

In particular, to calculate the output values of the neural network 300, the input values are propagated through the neural network. In particular, the values of the nodes 320, . . . , 332 of the (n+1)-th layer 310, . . . , 313 can be calculated based on the values of the nodes 320, . . . , 332 of the n-th layer 310, . . . , 313 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 310 are given by the input of the neural network 300, wherein values of the first hid-den layer 311 can be calculated based on the values of the input layer 310 of the neural network, wherein values of the second hidden layer 312 can be calculated based in the values of the first hidden layer 311, etc.

In order to set the values W^{(m,n)}i,j for the edges, the neural network 300 has to be trained using training data. In particular, training data comprises training input data and training output data (denoted as tᵢ). For a training step, the neural network 300 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 300 (backpropagation algorithm). In particular, the weights are changed according to

$${{w}^{′}}^{{( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 313, wherein f′ is the first derivative of the activation function, and t⁽ⁿ⁺¹⁾ⱼ is the comparison training value for the j-th node of the output layer 313.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

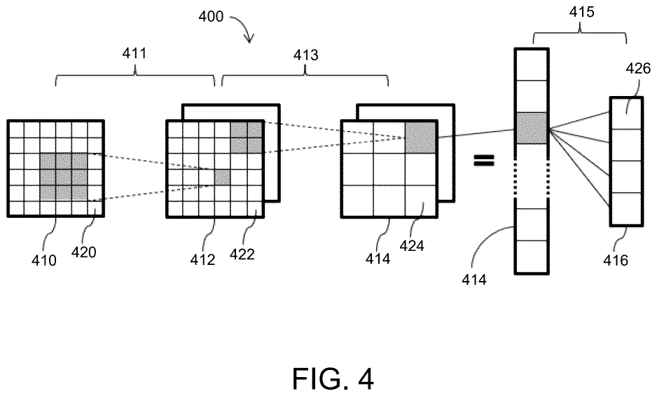

FIG. 4 shows an embodiment of a convolutional neural network 400 that may be used to implement one or more machine learning models described herein. In the displayed embodiment, the convolutional neural network 400 comprises an input node layer 410, a convolutional layer 411, a pooling layer 413, a fully connected layer 414 and an output node layer 416, as well as hidden node layers 412, 414. Alternatively, the convolutional neural network 400 can comprise several convolutional layers 411, several pooling layers 413 and several fully connected layers 415, as well as other types of layers. The order of the layers can be chosen arbitrarily, usually fully connected layers 415 are used as the last layers before the output layer 416.

In particular, within a convolutional neural network 400 nodes 420, 422, 424 of a node layer 410, 412, 414 can be considered to be arranged as a d-dimensional matrix or as a d-dimensional image. In particular, in the two-dimensional case the value of the node 420, 422, 424 indexed with i and j in the n-th node layer 410, 412, 414 can be denoted as x(n)[i, j]. However, the arrangement of the nodes 420, 422, 424 of one node layer 410, 412, 414 does not have an effect on the calculations executed within the convolutional neural network 400 as such, since these are given solely by the structure and the weights of the edges.

A convolutional layer 411 is a connection layer between an anterior node layer 410 (with node values x(n-1)) and a posterior node layer 412 (with node values x(n)). In particular, a convolutional layer 411 is characterized by the structure and the weights of the incoming edges forming a convolution operation based on a certain number of kernels. In particular, the structure and the weights of the edges of the convolutional layer 411 are chosen such that the values x(n) of the nodes 422 of the posterior node layer 412 are calculated as a convolution x(n)=K*x(n-1) based on the values x(n-1) of the nodes 420 anterior node layer 410, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 420, 422 (e.g., a 3×3 matrix, or a 5×5 matrix). In particular, this implies that the weights of the edges in the convolution layer 411 are not independent, but chosen such that they produce said convolution equation. In particular, for a kernel being a 3×3 matrix, there are only 9 independent weights (each entry of the kernel matrix corresponding to one independent weight), irrespectively of the number of nodes 420, 422 in the anterior node layer 410 and the posterior node layer 412.

In general, convolutional neural networks 400 use node layers 410, 412, 414 with a plurality of channels, in particular, due to the use of a plurality of kernels in convolutional layers 411. In those cases, the node layers can be considered as (d+1)-dimensional matrices (the first dimension indexing the channels). The action of a convolutional layer 411 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 410, x(n)b corresponds to the b-th channel of the posterior node layer 412 and K_{a,b} corresponds to one of the kernels. If a convolutional layer 411 acts on an anterior node layer 410 with A channels and outputs a posterior node layer 412 with B channels, there are A-B independent d-dimensional kernels K_{a,b}.

In general, in convolutional neural networks 400 activation functions are used. In this embodiment ReLU (acronym for “Rectified Linear Units”) is used, with R(z)=max(0, z), so that the action of the convolutional layer 411 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 410 comprises 36 nodes 420, arranged as a two-dimensional 6×6 matrix. The first hidden node layer 412 comprises 72 nodes 422, arranged as two two-dimensional 6×6 matrices, each of the two matrices being the result of a convolution of the values of the input layer with a 3×3 kernel within the convolutional layer 411. Equivalently, the nodes 422 of the first hidden node layer 412 can be interpreted as arranged as a three-dimensional 2×6×6 matrix, wherein the first dimension correspond to the channel dimension.

The advantage of using convolutional layers 411 is that spatially local correlation of the input data can exploited by enforcing a local connectivity pattern between nodes of adjacent layers, in particular by each node being connected to only a small region of the nodes of the preceding layer.

A pooling layer 413 is a connection layer between an anterior node layer 412 (with node values x(n-1)) and a posterior node layer 414 (with node values x(n)). In particular, a pooling layer 413 can be characterized by the structure and the weights of the edges and the activation function forming a pooling operation based on a non-linear pooling function f. For example, in the two-dimensional case the values x(n) of the nodes 424 of the posterior node layer 414 can be calculated based on the values x(n-1) of the nodes 422 of the anterior node layer 412 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 413 the number of nodes 422, 424 can be reduced, by re-placing a number d1·d2 of neighboring nodes 422 in the anterior node layer 412 with a single node 422 in the posterior node layer 414 being calculated as a function of the values of said number of neighboring nodes. In particular, the pooling function f can be the max-function, the average or the L2-Norm. In particular, for a pooling layer 413 the weights of the incoming edges are fixed and are not modified by training.

The advantage of using a pooling layer 413 is that the number of nodes 422, 424 and the number of parameters is reduced. This leads to the amount of computation in the network being reduced and to a control of overfitting.

In the displayed embodiment, the pooling layer 413 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 400 are fully connected layers 415. A fully connected layer 415 is a connection layer between an anterior node layer 414 and a posterior node layer 416. A fully connected layer 413 can be characterized by the fact that a majority, in particular, all edges between nodes 414 of the anterior node layer 414 and the nodes 416 of the posterior node layer are present, and wherein the weight of each of these edges can be adjusted individually.

In this embodiment, the nodes 424 of the anterior node layer 414 of the fully connected layer 415 are displayed both as two-dimensional matrices, and additionally as non-related nodes (indicated as a line of nodes, wherein the number of nodes was reduced for a better presentability). This operation is also denoted as “flattening”. In this embodiment, the number of nodes 426 in the posterior node layer 416 of the fully connected layer 415 smaller than the number of nodes 424 in the anterior node layer 414. Alternatively, the number of nodes 426 can be equal or larger.

Furthermore, in this embodiment the Softmax activation function is used within the fully connected layer 415. By applying the Softmax function, the sum the values of all nodes 426 of the output layer 416 is 1, and all values of all nodes 426 of the output layer 416 are real numbers between 0 and 1. In particular, if using the convolutional neural network 400 for categorizing input data, the values of the output layer 416 can be interpreted as the probability of the input data falling into one of the different categories.

In particular, convolutional neural networks 400 can be trained based on the backpropagation algorithm. For preventing overfitting, methods of regularization can be used, e.g., dropout of nodes 420, . . . , 424, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m-2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

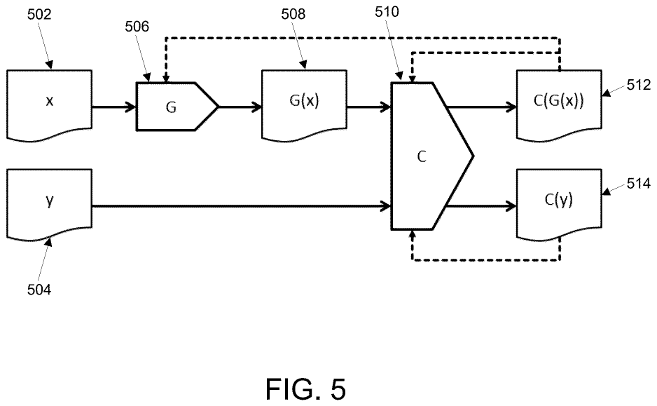

FIG. 5 shows a data flow diagram according to an embodiment for using a generative adversarial network for creating synthetic output data G(x) 508 based on input data x 502 that is indistinguishable from real output data y 504, in accordance with one or more embodiments. The synthetic output data G(x) 508 has the same structure as the real output data y 504, but its content is not derived from real world data.

The generative adversarial network comprises a generator function G 506 and a classifier function C 510 which are trained jointly. The task of the generator function G 506 is to provide realistic synthetic output data G(x) 508 based on input data x 502, and the task of the classifier function C 510 is to distinguish between real output data y 504 and synthetic output data G(x) 508. In particular, the output of the classifier function C 510 is a real number between 0 and 1 corresponding to the probability of the input value being real data, so that an ideal classifier function would calculate an output value of C(y) 514≈1 for real data y 504 and C(G(x)) 512≈0 for synthetic data G(x) 508.

Within the training process, parameters of the generator function G 506 are adapted so that the synthetic output data G(x) 508 has the same characteristics as real output data y 504, so that the classifier function C 510 cannot distinguish between real and synthetic data anymore. At the same time, parameters of the classifier function C 510 are adapted so that it distinguishes between real and synthetic data in the best possible way. Here, the training relies on pairs comprising input data x 502 and the corresponding real output data y 504. Within a single training step, the generator function G 506 is applied to the input data x 502 for generating synthetic output data G(x) 508. Furthermore, the classifier function C 510 is applied to the real output data y 504 for generating a first classification result C(y) 514. Additionally, the classifier function C 510 is applied to the synthetic output data G(x) 508 for generating a second classification result C(G(x)) 512.

Adapting the parameters of the generative function G 506 and the classifier function C 510 is based on minimizing a cost function by using the backpropagation algorithm, respectively. In this embodiment, the cost function K_{C} for the classifier function C 510 is K_{C} ∝−BCE(C(y), 1)−BCE(C(G(x)), 0), wherein BCE denotes the binary cross entropy defined as BCE(z, z′)=z′·log(z)+(1−z′)·log(1−z). By using this cost function, both wrongly classifying real output data as synthetic (indicated by C(y)≈0) and wrongly classifying synthetic output data as real (indicated as C(G(x)) 512≈1) increases the cost function K_{C} to be minimized. Furthermore, the cost function K_{G} for the generator function G 506 is K_{G} ∝−BCE(C(G(x)), 1)=−log (C(G(x)). By using this cost function, correctly classified synthetic output data (indicated as C(G(x)) 512≈0) leads to an increase of the cost function K_{G} to be minimized.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

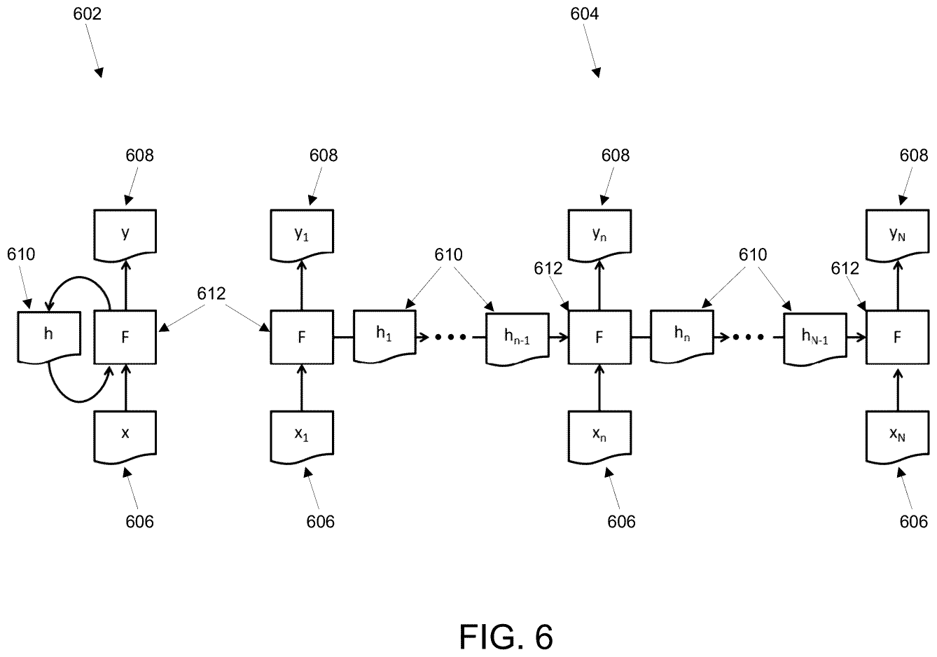

FIG. 6 shows the schematic structure of a recurrent machine learning model F, both in a recurrent representation 602 and in an unfolded representation 604, that may be used to implement one or more machine learning models described herein. The recurrent machine learning model takes as input several input datasets x, x₁, . . . , x_{N} 606 and creates a corresponding set of output datasets y, y₁, . . . , y_{N} 608. Furthermore, the output depends on a so-called hidden vector h, h₁, . . . , h_{N} 610, which implicitly comprises information about input datasets previously used as input for the recurrent machine learning model F 612. By using these hidden vectors h, h₁, . . . , h_{N} 610, a sequentiality of the input datasets can be leveraged.

In a single step of the processing, the recurrent machine learning model F 612 takes as input the hidden vector hₙ₋₁ created within the previous step and an input dataset xₙ. Within this step, the recurrent machine learning model F generates as output an updated hidden vector ha and an output dataset yₙ. In other words, one step of processing calculates (yₙ, hₙ)=F(xₙ, hₙ₋₁), or by splitting the recurrent machine learning model F 612 into a part F(y) calculating the output data and F(h) calculating the hidden vector, one step of processing calculates yₙ=F(y)(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 612 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 2. Certain steps or functions of the methods and workflows described herein, including one or more of the steps or functions of FIG. 1 or 2, may be performed by a server or by another processor in a network-based cloud-computing system. Certain steps or functions of the methods and workflows described herein, including one or more of the steps of FIG. 1 or 2, may be performed by a client computer in a network-based cloud computing system. The steps or functions of the methods and workflows described herein, including one or more of the steps of FIG. 1 or 2, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 702 that may be used to implement systems, apparatuses, and methods described herein is depicted in FIG. 7. Computer 702 includes a processor 704 operatively coupled to a data storage device 712 and a memory 710. Processor 704 controls the overall operation of computer 702 by executing computer program instructions that define such operations. The computer program instructions may be stored in data storage device 712, or other computer readable medium, and loaded into memory 710 when execution of the computer program instructions is desired. Thus, the method and workflow steps or functions of FIG. 1 or 2 can be defined by the computer program instructions stored in memory 710 and/or data storage device 712 and controlled by processor 704 executing the computer program instructions. For example, the computer program instructions can be implemented as computer executable code programmed by one skilled in the art to perform the method and workflow steps or functions of FIG. 1 or 2. Accordingly, by executing the computer program instructions, the processor 704 executes the method and workflow steps or functions of FIG. 1 or 2. Computer 702 may also include one or more network interfaces 706 for communicating with other devices via a network. Computer 702 may also include one or more input/output devices 708 that enable user interaction with computer 702 (e.g., display, keyboard, mouse, speakers, buttons, etc.).

Processor 704 may include both general and special purpose microprocessors, and may be the sole processor or one of multiple processors of computer 702. Processor 704 may include one or more central processing units (CPUs), for example. Processor 704, data storage device 712, and/or memory 710 may include, be supplemented by, or incorporated in, one or more application-specific integrated circuits (ASICs) and/or one or more field programmable gate arrays (FPGAs).

Data storage device 712 and memory 710 each include a tangible non-transitory computer readable storage medium. Data storage device 712, and memory 710, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 708 may include peripherals, such as a printer, scanner, display screen, etc. For example, input/output devices 708 may include a display device such as a cathode ray tube (CRT) or liquid crystal display (LCD) monitor for displaying information to the user, a keyboard, and a pointing device such as a mouse or a trackball by which the user can provide input to computer 702.

An image acquisition device 714 can be connected to the computer 702 to input image data (e.g., medical images) to the computer 702. It is possible to implement the image acquisition device 714 and the computer 702 as one device. It is also possible that the image acquisition device 714 and the computer 702 communicate wirelessly through a network. In a possible embodiment, the computer 702 can be located remotely with respect to the image acquisition device 714.

Any or all of the systems, apparatuses, and methods discussed herein may be implemented using one or more computers such as computer 702.

One skilled in the art will recognize that an implementation of an actual computer or computer system may have other structures and may contain other components as well, and that FIG. 7 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving one or more 3D (three-dimensional) medical images each comprising a plurality of 2D (two-dimensional) slices; extracting a first set of features from the plurality of 2D slices of the one or more 3D medical images using a machine learning based encoding network; resampling each respective feature of the first set of features based on a spatial location of one or more pixels of the 2D slices from which the respective feature was extracted; encoding the resampled first set of features into a second set of features; performing a medical imaging analysis task based on the second set of features; and outputting results of the medical imaging analysis task.

Illustrative embodiment 2. The computer-implemented method of illustrative embodiment 1, wherein the one or more 3D medical images are of at least one of different resolutions, different acquisition orientations, or different domains.

Illustrative embodiment 3. The computer-implemented method of any one of illustrative embodiments 1-2, wherein resampling each respective feature of the first set of features based on a spatial location of one or more pixels of the 2D slices from which the respective feature was extracted comprises: resampling each respective feature of the first set of features onto a 2D uniform pixel grid to form 2D images in a feature space.

Illustrative embodiment 4. The computer-implemented method of illustrative embodiment 3, wherein resampling each respective feature of the first set of features based on a spatial location of one or more pixels of the 2D slices from which the respective feature was extracted further comprises: stacking the 2D images in the feature space to form one or more 3D isotropic images in the feature space.

Illustrative embodiment 5. The computer-implemented method of any one of illustrative embodiments 1-4, wherein the spatial location of the one or more pixels of the 2D slices comprises coordinates of the one or more pixels.

Illustrative embodiment 6. The computer-implemented method of any one of illustrative embodiments 1-5, wherein resampling each respective feature of the first set of features based on a spatial location of one or more pixels of the 2D slices from which the respective feature was extracted comprises: encoding the first set of features with positional embeddings.

Illustrative embodiment 7. The computer-implemented method of any one of illustrative embodiments 1-6, wherein resampling each respective feature of the first set of features based on a spatial location of one or more pixels of the 2D slices from which the respective feature was extracted comprises: encoding the first set of features with modality embeddings.

Illustrative embodiment 8. The computer-implemented method of any one of illustrative embodiments 1-7, wherein encoding the resampled first set of features into a second set of features comprises: encoding the resampled first set of features into the second set of features using at least one of a transformer encoder or a state space model.

Illustrative embodiment 9. The computer-implemented method of any one of illustrative embodiments 1-8, wherein performing a medical imaging analysis task based on the second set of features comprises: performing the medical imaging analysis task using at least one of a transformer encoder or a state space model.

Illustrative embodiment 10. An apparatus comprising: means for receiving one or more 3D (three-dimensional) medical images each comprising a plurality of 2D (two-dimensional) slices; means for extracting a first set of features from the plurality of 2D slices of the one or more 3D medical images using a machine learning based encoding network; means for resampling each respective feature of the first set of features based on a spatial location of one or more pixels of the 2D slices from which the respective feature was extracted; means for encoding the resampled first set of features into a second set of features; means for performing a medical imaging analysis task based on the second set of features; and means for outputting results of the medical imaging analysis task.

Illustrative embodiment 11. The apparatus of illustrative embodiment 10, wherein the one or more 3D medical images are of at least one of different resolutions, different acquisition orientations, or different domains.

Illustrative embodiment 12. The apparatus of any one of illustrative embodiments 10-11, wherein the means for resampling each respective feature of the first set of features based on a spatial location of one or more pixels of the 2D slices from which the respective feature was extracted comprises: means for resampling each respective feature of the first set of features onto a 2D uniform pixel grid to form 2D images in a feature space.

Illustrative embodiment 13. The apparatus of any one of illustrative embodiments 10-12, wherein the means for resampling each respective feature of the first set of features based on a spatial location of one or more pixels of the 2D slices from which the respective feature was extracted further comprises: means for stacking the 2D images in the feature space to form one or more 3D isotropic images in the feature space.

Illustrative embodiment 14. The apparatus of any one of illustrative embodiments 10-14, wherein the spatial location of the one or more pixels of the 2D slices comprises coordinates of the one or more pixels.

Illustrative embodiment 15. A non-transitory computer-readable storage medium comprising instructions which, when executed by a computer, cause the computer to carry out operations comprising: receiving one or more 3D (three-dimensional) medical images each comprising a plurality of 2D (two-dimensional) slices; extracting a first set of features from the plurality of 2D slices of the one or more 3D medical images using a machine learning based encoding network; resampling each respective feature of the first set of features based on a spatial location of one or more pixels of the 2D slices from which the respective feature was extracted; encoding the resampled first set of features into a second set of features; performing a medical imaging analysis task based on the second set of features; and outputting results of the medical imaging analysis task.

Illustrative embodiment 16. The non-transitory computer-readable storage medium of illustrative embodiment 15, wherein the one or more 3D medical images are of at least one of different resolutions, different acquisition orientations, or different domains.

Illustrative embodiment 17. The non-transitory computer-readable storage medium of any one of illustrative embodiments 15-16, wherein resampling each respective feature of the first set of features based on a spatial location of one or more pixels of the 2D slices from which the respective feature was extracted comprises: encoding the first set of features with positional embeddings.

Illustrative embodiment 18. The non-transitory computer-readable storage medium of any one of illustrative embodiments 15-17, wherein resampling each respective feature of the first set of features based on a spatial location of one or more pixels of the 2D slices from which the respective feature was extracted comprises: encoding the first set of features with modality embeddings.

Illustrative embodiment 19. The non-transitory computer-readable storage medium of any one of illustrative embodiments 15-18, wherein encoding the resampled first set of features into a second set of features comprises: encoding the resampled first set of features into the second set of features using at least one of a transformer encoder or a state space model.

Illustrative embodiment 20. The non-transitory computer-readable storage medium of any one of illustrative embodiments 15-19, wherein performing a medical imaging analysis task based on the second set of features comprises: performing the medical imaging analysis task using at least one of a transformer encoder or a state space model.

1. A computer-implemented method comprising:
receiving one or more 3D (three-dimensional) medical images each comprising a plurality of 2D (two-dimensional) slices;
extracting a first set of features from the plurality of 2D slices of the one or more 3D medical images using a machine learning based encoding network;
resampling each respective feature of the first set of features based on a spatial location of one or more pixels of the 2D slices from which the respective feature was extracted;
encoding the resampled first set of features into a second set of features;
performing a medical imaging analysis task based on the second set of features; and
outputting results of the medical imaging analysis task.

2. The computer-implemented method of claim 1, wherein the one or more 3D medical images are of at least one of different resolutions, different acquisition orientations, or different domains.

3. The computer-implemented method of claim 1, wherein resampling each respective feature of the first set of features based on a spatial location of one or more pixels of the 2D slices from which the respective feature was extracted comprises:
resampling each respective feature of the first set of features onto a 2D uniform pixel grid to form 2D images in a feature space.

4. The computer-implemented method of claim 3, wherein resampling each respective feature of the first set of features based on a spatial location of one or more pixels of the 2D slices from which the respective feature was extracted further comprises:
stacking the 2D images in the feature space to form one or more 3D isotropic images in the feature space.

5. The computer-implemented method of claim 1, wherein the spatial location of the one or more pixels of the 2D slices comprises coordinates of the one or more pixels.

6. The computer-implemented method of claim 1, wherein resampling each respective feature of the first set of features based on a spatial location of one or more pixels of the 2D slices from which the respective feature was extracted comprises:
encoding the first set of features with positional embeddings.

7. The computer-implemented method of claim 1, wherein resampling each respective feature of the first set of features based on a spatial location of one or more pixels of the 2D slices from which the respective feature was extracted comprises:
encoding the first set of features with modality embeddings.

8. The computer-implemented method of claim 1, wherein encoding the resampled first set of features into a second set of features comprises:
encoding the resampled first set of features into the second set of features using at least one of a transformer encoder or a state space model.

9. The computer-implemented method of claim 1, wherein performing a medical imaging analysis task based on the second set of features comprises:
performing the medical imaging analysis task using at least one of a transformer encoder or a state space model.

10. An apparatus comprising:
means for receiving one or more 3D (three-dimensional) medical images each comprising a plurality of 2D (two-dimensional) slices;
means for extracting a first set of features from the plurality of 2D slices of the one or more 3D medical images using a machine learning based encoding network;
means for resampling each respective feature of the first set of features based on a spatial location of one or more pixels of the 2D slices from which the respective feature was extracted;
means for encoding the resampled first set of features into a second set of features;
means for performing a medical imaging analysis task based on the second set of features; and
means for outputting results of the medical imaging analysis task.

11. The apparatus of claim 10, wherein the one or more 3D medical images are of at least one of different resolutions, different acquisition orientations, or different domains.

12. The apparatus of claim 10, wherein the means for resampling each respective feature of the first set of features based on a spatial location of one or more pixels of the 2D slices from which the respective feature was extracted comprises:
means for resampling each respective feature of the first set of features onto a 2D uniform pixel grid to form 2D images in a feature space.

13. The apparatus of claim 12, wherein the means for resampling each respective feature of the first set of features based on a spatial location of one or more pixels of the 2D slices from which the respective feature was extracted further comprises:
means for stacking the 2D images in the feature space to form one or more 3D isotropic images in the feature space.

14. The apparatus of claim 10, wherein the spatial location of the one or more pixels of the 2D slices comprises coordinates of the one or more pixels.

15. A non-transitory computer-readable storage medium comprising instructions which, when executed by a computer, cause the computer to carry out operations comprising:
receiving one or more 3D (three-dimensional) medical images each comprising a plurality of 2D (two-dimensional) slices;
extracting a first set of features from the plurality of 2D slices of the one or more 3D medical images using a machine learning based encoding network;
resampling each respective feature of the first set of features based on a spatial location of one or more pixels of the 2D slices from which the respective feature was extracted;
encoding the resampled first set of features into a second set of features;
performing a medical imaging analysis task based on the second set of features; and
outputting results of the medical imaging analysis task.

16. The non-transitory computer-readable storage medium of claim 15, wherein the one or more 3D medical images are of at least one of different resolutions, different acquisition orientations, or different domains.

17. The non-transitory computer-readable storage medium of claim 15, wherein resampling each respective feature of the first set of features based on a spatial location of one or more pixels of the 2D slices from which the respective feature was extracted comprises:
encoding the first set of features with positional embeddings.

18. The non-transitory computer-readable storage medium of claim 15, wherein resampling each respective feature of the first set of features based on a spatial location of one or more pixels of the 2D slices from which the respective feature was extracted comprises:
encoding the first set of features with modality embeddings.

19. The non-transitory computer-readable storage medium of claim 15, wherein encoding the resampled first set of features into a second set of features comprises:
encoding the resampled first set of features into the second set of features using at least one of a transformer encoder or a state space model.

20. The non-transitory computer-readable storage medium of claim 15, wherein performing a medical imaging analysis task based on the second set of features comprises:
performing the medical imaging analysis task using at least one of a transformer encoder or a state space model.
